(19) United States
(12) Patent Application Publication
(10) Pub. No.: US 2023/0309457 A1
(43) Pub. Date: Oct. 5, 2023

(54) PROPAGATING MATERIAL FOR SWEET POTATO

(71) Applicant: PIETERS JOSEPH & LUC BV, Staden (BE)

(72) Inventors: Christophe PIETERS, Staden (BE); Luc PIETERS, Staden (BE)

(21) Appl. No.: 17/999,975

(22) PCT Filed: May 25, 2021
(86) PCT No.: PCT/IB2021/054512

§ 371 Date: Nov. 28, 2022

(30) Foreign Application Priority Data
May 26, 2020 (BE) 2020/5364

(51) Int. Cl. A01G 2/10; A01G 22/25

(52) U.S. Cl. CPC A01G 2/10; A01G 22/25

(57) ABSTRACT

In a first aspect, the invention relates to a method for obtaining a sweet potato propagating material. In a second and third aspect, the invention relates to sweet potato propagating material and a plurality thereof.

### TECHNICAL FIELD

The invention relates to a method for obtaining propagating material for sweet potato, the propagating material and a packaged plurality thereof.

### PRIOR ART

Methods for obtaining vegetative propagating material for sweet potato are known in the art.

Traditionally, sweet potato is propagated vegetatively by means of slips, i.e. sprouted cuttings of at least 20 cm. The production of slips requires a large surface area, is time consuming and results in non-uniform plants with unbalanced above-ground growth. In addition, it is difficult to plant slips semi-automatically or fully automatically. Planting out manually is the norm here.

There is a need in the prior art for a method for propagating sweet potato quickly and with high productivity on a small area.

In particular, there is a need for such a method that yields planting stock that can be easily planted out and shows good growth after planting out.

The present invention aim to resolve at least some of the above-mentioned problems.

### SUMMARY OF THE INVENTION

In a first aspect, the invention relates to a method for obtaining a sweet potato propagating material according to claim 1.

In a second aspect, the invention relates to a sweet potato propagating material according to claim 14.

In a third aspect, the invention relates to a plurality of the sweet potato propagating material according to claim 16.

Preferred embodiments of the present invention are discussed in claims 2 to 13, 15 and 17 as well as throughout the specification.

The invention provides planting stock that is uniform and easy to plant out, which also shows good growth after planting out. Moreover, such planting stock can easily be planted out by machine, semi-automatically or fully automatically. In addition, the present invention also provides a method for high productivity propagation of sweet potato on a small substrate bed area.

Further advantages, embodiments and preferred embodiments of the invention are discussed below in the detailed description and examples.

### DETAILED DESCRIPTION

In a first aspect, the invention relates to a method for obtaining a sweet potato propagating material. In a second and third aspect, the invention relates to a sweet potato propagating material as well as a plurality thereof. The invention was summarised in the section provided for this purpose. In what follows, the invention is described in detail, and preferred embodiments are explained.

Unless otherwise defined, all terms used in the description of the invention, including technical and scientific terms, have the meaning as commonly understood by a person skilled in the art to which the invention pertains. For a better understanding of the description of the invention, the following terms are explained explicitly.

In this document, ‘a’ and ‘the’ refer to both the singular and the plural, unless the context presupposes otherwise. For example, ‘a segment’ means one or more segments.

When the term ‘around’ or ‘about’ is used in this document with a measurable quantity, a parameter, a duration or moment, and the like, then variations are meant of approx. 20% or less, preferably approx. 10% or less, more preferably approx. 5% or less, even more preferably approx. 1% or less, and even more preferably approx. 0.1% or less than and of the quoted value. However, it must be understood that the value of a quantity used where the term ‘about’ or ‘around’ is used, is itself specifically disclosed. The terms ‘comprise’, ‘comprising’, ‘consist of’, ‘consisting of’, ‘provided with’, ‘have’, ‘having’, ‘include’, ‘including’, ‘contain’, ‘containing’ are synonyms and are inclusive or open terms that indicate the presence of what follows, and which do not exclude or prevent the presence of other characteristics, elements, members, steps, as known from or disclosed in the prior art.

Quoting numerical intervals by endpoints comprises all integers, fractions and/or real numbers between the endpoints, these endpoints included.

In a first aspect, the invention relates to a method for obtaining a sweet potato propagating material. The method preferably comprises taking a cutting from a stem of a growing sweet potato plant. The cutting is preferably a stem section provided with an apical bud and more preferably at least one node. Preferably, a length of the stem section between an apical end of the apical bud and a basal end is between 2 and 10 cm.

In a second aspect, the invention relates to a sweet potato propagating material. The propagating material is preferably a cutting obtained from a growing sweet potato plant.

The cutting is preferably a stem section provided with an apical bud and more preferably at least one node. Preferably, a length of the stem section between an apical end of the apical bud and a basal end is between 2 and 10 cm. Preferably, the propagating material according to the second aspect is obtained according to the first aspect of the invention.

In a third aspect, the invention relates to a plurality of the sweet potato propagating material according to a second aspect of the invention. Preferably, the plurality of propagating material is packaged in a bundle.

A person of ordinary skill in the art will appreciate that the method for obtaining the sweet potato propagating material according to a first aspect of the invention can be used to obtain the propagating material or a plurality thereof according to a second and third aspect of the invention. In what follows, the three aspects of the present invention are therefore treated together. Furthermore, each feature described above as well as below, may relate to any of the three aspects, even if the feature is described in conjunction with a specific aspect of the present invention.

The object of the invention is a sweet potato propagating material and a packaged plurality thereof suitable for storage and/or transport. In particular, a propagating material for obtaining sweet potato with high uniformity, rapid growth and high tuber yield. In particular, a propagating material extremely suitable for planting out mechanically. It is also the object of the present invention to provide a method for obtaining propagating material for sweet potatoes with high productivity. In particular, a method extremely suitable for production on a small surface, in particular a small substrate bed area.

‘Sweet potato’ as used herein refers to a term known in the art designating a plant of the Convolvulaceae family with the scientific name Ipomoea batatas. In addition, sweet potato herein may refer to any cultivar known in the art. Examples of cultivars include Alabama Purple, Allgold, Arkansas Red, Beauregard, Bunch Porto Ricos, Carolina Bunch, Centennial, Cordner's Red, Covington, Darby, Evangeline, Excel, Georgia Jet, Georgia Red, Hernandez, Jewel, Kotobuki, Molokai Purple, Nancy Hall, New Jewell, Norton, O'Henry, Okinawa, Okinawa Purple, Oklahoma Heirloom, Oriental, Red Garnet, Red Jewel, Red Resisto, Porto Rican, Regal, Resisto, Satsuma, Southern Delite, Sumor, Vardaman, White Triumph and Yamiamo.

Sweet potato cultivars can be classified by growth habit. The growth habits are divided into erect, semi-erect, flat and very flat. The term ‘erected’ in this context denotes a shoot with a substantially vertical stem. The term ‘flat’ in this context denotes a shoot in which the stems grow substantially parallel to a soil surface. Preferably, the sweet potato plant is a plant with an erect or semi-erect growth habit, most preferably an erect growth habit. Erect and semi-erect growth habits are particularly advantageous for growing sweet potato plants in a small area. A minimal growing area is advantageous for plants for cutting production, especially cutting production in a greenhouse in a cold to temperate climate.

Preferably, the sweet potato is a Beauregard cultivar. Sweet potato plants of the Beauregard cultivar are, among other things, characterised by an erect growth habit.

‘Propagating material’ or ‘plant propagating material’, as used herein, refers to a term known in the art that denotes a generative or vegetative plant part that can be used for propagation of a plant, as well as a sprouted or young plant obtained from such generative or vegetative plant part. Propagating materials accordingly include seeds, fruits, cuttings, roots, tubers, bulbs, rhizomes or other plant parts, as well as planting stock. ‘Planting stock’, as used herein, refers to a term known in the art which denotes a plant intended for planting out. Until the moment that a plant is definitively planted out in, for example, a field for production, it should be understood as planting stock. The majority of cultivated, planted or traded plants known in the art are planting stock. In particular, planting stock herein refers to a rooted cutting, whether or not provided with substrate.

The vegetative propagation of sweet potato by means of cuttings is known in the art. Vegetative propagation has the advantage that cuttings can be selected based on the health of a mother plant. Mother plants that do not show disease symptoms such as a virus infection or a nutrient deficiency are preferably selected. Consequently, healthy planting stock can be obtained.

The propagating material according to the present invention is obtained by taking a cutting from a stem of a growing sweet potato plant. The cutting is a stem section obtained from the full-grown plant. The stem part can be obtained by taking this cutting from a stem of a full-grown sweet potato plant. This cutting can be taken either manually or mechanically. The cutting can be taken by pulling, cutting, shearing, folding or cracking the stem section of the stem from the full-grown plant. Preferably, the sweet potato plant cutting is split by hand, scissors or a knife. For example, taking a cutting manually can be performed by a worker. The scissors and blade can be powered manually or mechanically.

Preferably, the cutting of the stem section is taken from a stem of the full-grown sweet potato plant having a length shorter than 50 cm, more preferably shorter than 40 cm, more preferably shorter than 30 cm and most preferably shorter than 20 cm. A length of a stem is to be understood as a length between an apical end of this stem and a node or tuber to which this stem is connected. Stems with a length shorter than 50 cm and in particular shorter than 20 cm show no or only limited lignification. Such stem sections will therefore more easily form roots since the roots do not have to penetrate a lignified layer in a stem. Preferably, the stem section cutting is taken at a uniform height from the full-grown sweet potato plants. A height of a full-grown sweet potato plant is to be understood as a length between an apical end of a stem piece cutting and a soil surface. Plants with an erect growth habit are preferably used to optimise the taking of the cutting at a uniform height both manually and mechanically. Planting stock and plants grown therefrom ready for harvest obtained from cuttings split off at a uniform height are characterised by more uniform growth and yield.

Preferably, the stem section of the present invention is provided with an apical bud. Planting stock obtained from stem sections with an apical bud will show a strong apical dominance. The apex of such planting stock will consequently show a stronger development than its lateral branches. Such high apical dominance shortens, among other things, the time it takes for the planting stock to reach the minimum size required for planting out in a field. In addition, such planting stock will show better growth once planted out, especially when planted out by machine. After all, during mechanical transplanting, the apical ends of planting stock obtained from stem sections without apical buds will be frequently covered with soil. This will strongly inhibit the growth of such planting stock. Due to the strong development of the apex of planting stock obtained from stem sections with apical buds, they will be covered with soil less frequently, and when covered show a faster growth compared to planting stock not obtained according to the present invention.

Preferably, the stem section of the present invention is further provided with at least one node. The stem section may or may not have one, two, three, four, five, six, seven, eight, nine, ten, eleven, twelve, thirteen, fourteen, fifteen, sixteen, seventeen, eighteen, nineteen or twenty nodes. More preferably, the stem section is provided with at most five nodes. Even more preferably, the stem section is provided with two or three nodes. Most preferably, the stem section is provided with three nodes. Sweet potato has an alternate phyllotaxis (or leaf arrangement), i.e. a leaf arrangement in which the leaves of a stem are arranged in a spiral around this stem at a substantially even distance from each other and in which the portion of the spiral turn between the various successive leaves forms a fraction. In particular, sweet potato has a ⅖ phyllotaxis, i.e. a leaf arrangement in which five leaves are arranged in a spiral extending over two revolutions. As a result, one leaf is provided per node on a stem and leaves on successive nodes are provided at an angle of approximately 144° to each other. A stem section according to the present invention provided with two or three nodes and in particular three nodes is therefore extremely advantageous for growth into planting stock, since it will receive light optimally, i.e. on all sides around and substantially uninterrupted.

Preferably, the stem section according to the present invention has a length between an apical end of the apical bud and a basal end of between 2 and 10 cm, more preferably between 3 and 8 cm, still more preferably between 4 and 7 cm and most preferably between 5 and 6 cm. Cutting off apical ends of a stem of the full-grown sweet potato plant breaks the apical dominance of this stem. Breaking the apical dominance stimulates the emergence of lateral buds of this stem of the sweet potato plant. This results in a sweet potato plant with greater lateral branching. The inventors note that sweet potato plants having such lateral branching greatly increases the productivity of the method for obtaining the cuttings. In particular, the inventors note that by taking cuttings from stem sections with a length between 2 and 10 cm and more in particular 4 and 7 cm, the number of stems formed suitable for cutting to form a stem section according to the present invention is maximised. The inventors further note that planting stock and plants grown therefrom ready for harvest obtained with cuttings with a length between 2 and 10 cm, in particular between 3 and 8 cm and more particularly between 4 and 7 cm, are characterised by a more uniform growth and yield.

‘Apical bud’, ‘terminal bud’ or ‘end bud’, as used herein, refers to a term known in the art that denotes a bud provided at the end of a stem or shoot of a plant. The stem or shoot can refer to a main stem or main shoot as well as a lateral stem or lateral shoot. The apical bud of a sweet potato plant comprises a stem growing point.

‘Node’, ‘nodus’ or ‘joint’, as used herein, refers to a term known in the art which designates a location on a stem of a plant or shoot where a leaf or sub-stem is connected to this stem.

‘Basal end’, as used herein, refers to a term herein denoting the bottom end of a stem section of a sweet potato plant, i.e. where the stem section is cut from a stem of a full-grown sweet potato plant.

The stem section according to the present invention will form roots substantially at the level of a node. Root formation can be initiated spontaneously in moist conditions, for example when the stem section is exposed to a relative humidity higher than 60% for several days. These conditions can arise, for example, during transport by respiration of a plurality of stem sections in sealed or semi-sealed packages. The sweet potato cutting is preferably rooted by placing the basal end of the stem section in a substrate material. As a result, roots will be formed at the basal end of the stem piece and a rooted cutting can be obtained. It is not a requirement that the cutting be root-free at this time. For example, aerial roots may have been previously formed during transport. A rooted cutting is advantageous for planting out as it shows faster growth due to an already formed root system.

Preferably, the stem piece is watered and/or misted after placing the basal end in the substrate material. This promotes the rooting of the cutting and the growth of the resulting planting stock. Preferably, the substrate material is watered before the basal end of the stem piece is inserted into the substrate material. Preferably, a pit is formed in the substrate material for providing the basal end of the stem piece in the substrate material. Said pit may be formed manually or by machine. More preferably, the pit is watered before the basal end of the stem is placed into the substrate material. According to an alternative embodiment of the invention, the sweet potato cutting is rooted by providing the basal end in water, for example in a hydroculture system.

Preferably, the stem section is rooted at the level of the basal end. Since roots are mainly formed at a node of a stem section, the basal end of the stem section should preferably be provided as close as possible to the bottom node of the stem section. A bottom node herein is to be understood as the node provided on a stem section with the shortest distance from the basal end. A distance between the basal end and a bottom node of the stem section is preferably at most 2 cm, more preferably at most 1.8 cm, still further preferably at most 1.6 cm, still further preferably at most 1.4 cm, still further preferably at most 1.2 cm, still further preferably at most 1.0 cm, still further preferably at most 0.8 cm, still further preferably at most 0.6 cm, still further preferably at most 0.4 cm and at most preferably at most 0.2 cm.

Preferably, the basal end of the stem section is treated with an auxin before rooting. Auxins have a positive effect on the rooting percentage, the speed of rooting and the uniformity of the roots formed. Such auxin-treated planting stock takes a shorter time to reach the minimum size required for planting out in a field. Preferably, the auxin is one or more of auxin indolebutyric acid (IBA), indole acetic acid (IAA), and naphthyl acetic acid (NAA).

According to a preferred embodiment, the cutting is rooted by providing the basal end of the stem section in a substrate material provided in a plug. The plug herein may refer to any container or cell suitable for growing a cutting or other plant propagating material. Preferably, the plug has a volume comprised between 10 and 250 cm3, more preferably between 20 and 150 cm3 and most preferably between 30 and 100 cm3. The plug may be made of any material known in the art suitable for manufacturing a plant plug, such as, inter alia, polystyrene, plastic, wood, metal or paper. According to an alternative embodiment, the plug is a glue plug. A plurality of plugs may or may not be provided in a tray, such as, for example, per 40, 66, 84, 104 or 144. The plug may or may not be cone-shaped, beam-shaped, pyramid-shaped, cube-shaped or cylinder-shaped.

Preferably, the plug is a generally cylindrical plug with an open top. Such a cylinder shape promotes uniform root formation. More preferably, the plug is also provided with an open bottom. Such a cylindrical open plug prevents root accumulation during root formation. This increases the efficiency of the nutrient and water uptake from the soil when the rooted cutting is planted out.

Preferably, the plug is substantially made of biodegradable material. Such material has the advantage that the plug can be planted together with the cutting. More preferably, the plug is substantially made of a material that complies with one or more of EN13432, EN14995 and I5017088.

‘Biodegradable’ or ‘biologically degradable’ as used herein refers to a term known in the art that denotes a material that gradually decomposes and/or forms part of the common organic food chain within a measurable period of time when left alone in its natural condition.

Preferably, the plug is substantially made of paper. A paper plug isolates the substrate material, which promotes the root formation of a cutting. In addition, a paper plug is biodegradable and prevents root accumulation because formed roots can pierce the paper. More preferably, the paper is kraft paper. Kraft paper is porous, sturdy and biodegradable.

The full-grown sweet potato plant herein for obtaining the cutting may or may not refer to a sweet potato plant obtained from a generative plant part such as a sweet potato tuber or a vegetative plant part such as a cutting. Preferably, the full-grown sweet potato plant is grown from a generative plant part, in particular a sweet potato tuber. The inventors note that full-grown plants derived from tubers have greater cutting production productivity. In particular, the inventors note that when cuttings are taken according to the present invention with a length between 2 and 10 cm and more in particular with a length between 4 and 7 cm, the number of cuttings that can be produced per tuber increases considerably, compared to, for example, traditional slip production.

The sweet potato tuber for obtaining the full-grown sweet potato plant may refer to a propagation tuber (G0), a first generation root tuber (G1) or a second generation root tuber (G2), preferably the sweet potato tuber is a first generation (G1) root tuber.

Preferably, the sweet potato tuber from which the full-grown sweet potato plant is obtained is provided in a substrate bed. Such a substrate bed is usually beam-shaped with an open top. The bed is filled with a substrate material and a plurality of sweet potato tubers. The substrate material is preferably applied in two layers. A first layer of a substrate material is applied to the bottom of the bed at a depth of about 1 to 10 cm. The plurality of sweet potato tubers are applied to this first layer. A second layer of a substrate material is applied over these tubers at a depth of about 1 to 15 cm. Such substrate bed provides substrate material around each tuber.

According to a preferred embodiment, the sweet potato tuber is provided in a substrate bed at a temperature between 20 and 30° C., preferably about 25° C. The inventors note that at these temperatures, shoot formation and renewal are at their maximum. Consequently, at these temperatures, the number of cuttings produced will be at its maximum.

According to a preferred embodiment, the sweet potato tuber before growing into the full-grown sweet potato plant is subjected to a temperature between 27 and 40° C., preferably about 35° C., and a relative humidity of at least 80%, preferably about 90%, during a period of 4 to 16 days, preferably a period of about 8 days. Without pre-treatment, tubers will form shoots after a period of three to four weeks. This period can be shortened to two weeks with such pre-treatment. In addition, tubers subjected to such treatment will renew cut off stem sections more quickly.

According to a preferred embodiment, the substrate bed is provided with 25 to 400 sweet potato tubers per m2, more preferably 50 to 400 sweet potato tubers per m2 and most preferably about 100 sweet potato tubers per m2. The inventors note that a maximum number of about 1000 to 3000 cuttings per season can be formed per m2 substrate bed at a density of about 25 to 400 sweet potato tubers per m2 substrate bed. However, the optimal tuber density depends on an average tuber size and tuber shape. The average size and shape varies according to the cultivar used. In the case of the sweet potato cultivar Beauregard, the inventors note that an optimal tuber density is approximately 100 sweet potato tubers per m2 substrate bed.

According to a preferred embodiment, unwanted leaves are removed from the full-grown sweet potato plant. Preferably, wherein an undesired leaf has a length between a leaf base and an apical end of at least 3 cm, more preferably at least 3.5 cm, more preferably at least 4 cm, more preferably at least 4.5 cm and most preferably at least 5 cm. Removing unwanted leaves has the advantage that more uniform cuttings are obtained that will also show a more balanced growth. Removing unwanted leaves also ensures that new shoots are formed. Preferably, the unwanted leaves are removed at a leaf sheath. Taking the cutting in this matter minimises the lesion formed on the plant, allowing it to recover more quickly.

For transportation or storage, a plurality of the sweet potato propagating material according to the present invention may be packaged in a bundle.

‘Bundle’, as used herein, refers to a term denoting preferably a plurality of held together objects, more preferably a plurality of held together elongate objects, still more preferably a plurality of laterally held together elongate objects. For example, objects provided in a bundle can be held together by a packaging such as a foil or container and/or a lengthwise arranged connecting means such as a cable, ribbon, adhesive strip, rope or elastic band.

The packaging of the bundle herein may or may not be made of one or more materials selected from the group consisting of plastic, paper, cardboard, polystyrene, metal and glass. Preferably, the packaging of the bundle is substantially made of plastic and/or cardboard. Very preferably, the packaging of the bundle is substantially made of plastic.

According to a preferred embodiment, the packaging of the bundle is a perforated film, preferably a perforated plastic film. During storage or transport, the quality of the propagating material can decrease considerably. The most important processes here are respiration, transpiration and the formation of ethylene. In order to prevent the accumulation of moisture from respiration and transpiration as well as the accumulation of ethylene, the material of the packaging is preferably perforated. The accumulation of moisture can lead to the formation of mould. The accumulation of ethylene can lead to yellowing of the leaves, leaf fall and senescence.

In what follows, the invention is described by way of non-limiting examples illustrating the invention, and which are not intended to and should not be interpreted as limiting the scope of the invention.

### EXAMPLES

### Example 1

Example 1 relates to a method according to the present invention for obtaining cuttings for propagating sweet potato.

The sweet potatoes according to the present example are sweet potatoes of the Beauregard cultivar.

The steps for obtaining cuttings for propagating these potatoes are briefly explained below.

### Pre-Treating

First generation seed tubers (G1) are pre-treated by means of a curing treatment. The curing treatment subjects the tubers to a temperature of 35° C. and a relative humidity of 90% for a period of 8 days. This pre-treatment is necessary for wound healing of the tubers after harvesting. Such pre-treatment also promotes germination of the tubers.

### Bedding Down

After the pre-treatment, the treated tubers are bedded down in a substrate bed. A substrate material is applied to the bed in two layers. A first layer of the material is applied to the bottom of the bed with a depth of about 5 cm. The pre-treated seed tubers are applied to this first layer with a density of approximately 100 tubers per m². A second layer of the substrate material with a depth of approximately 10 cm is applied over these tubers. The bedded down tubers are watered frequently. After a period of about two weeks, shoots are formed.

### Propagating

The cuttings are obtained by taking stem section cuttings from the formed shoots. The stem sections have a length of about 5 cm and are provided with an apical bud and two or three nodes. Cuttings can be taken manually or mechanically. For example, a worker can sever the stem sections by hand, scissors or a knife. The scissors or blade can also be powered by a machine.

### Example 2

Example 2 relates to a comparative study between cuttings obtained according to Example 1 and slips with a length of about 25 cm.

Analogous to the cuttings from Example 1, the slips grown from G1 Beauregard seed tubers were bedded down with a density of about 100 tubers per m². About 2000 cuttings according to the present invention were harvested per m² substrate bed over a growth period of 2 months. However, only 400 slips per m² substrate bed were harvested over the same growing period.

The cuttings were rooted after harvest in a paper cylindrical plug with an open top and bottom. After a growth period of two weeks, these had a length of about 15 cm and were planted out by machine. The slips were manually planted out unrooted. Both varieties were planted out on potato ridges 75 cm and 30 cm apart in the row.

After a growth period of one month after planting out, the plants obtained from both varieties were evaluated on the basis of growth, balance and uniformity. The cuttings obtained according to the present invention showed a significant growth advantage as well as a more balanced above-ground growth and uniformity.

It is further noted that slips can only be harvested 2 to 3 times per season. However, cuttings according to Example 1 can be harvested continuously throughout the season.

In addition, cutting production according to Example 1 is extremely suitable for multi-layer cultivation, in particular for multi-layer germination in a substrate bed as well as multi-layer rooting of the obtained cuttings in, for example, a plug.

The present invention should not be construed as being limited to the embodiments described above and certain modifications or changes may be added to the examples described without having to re-evaluate the appended claims.

1. Method for obtaining a propagating material for sweet potato, the method comprising taking a cutting from a stem of a growing sweet potato plant, wherein the cutting is a stem section provided with an apical bud and at least one node and wherein a length of the stem section between an apical end of the apical bud and a basal end is between 2 and 10 cm.

2. The method according to claim 1, wherein the length between the apical end of the apical bud and the basal end is between 4 and 7 cm.

3. The method according to claim 1, wherein the stem section comprises at most five nodes, preferably two or three nodes, more preferably three nodes.

4. The method according to claim 1, wherein the sweet potato plant is grown from a sweet potato tuber.

5. The method according to claim 4, wherein the sweet potato tuber is provided in a substrate bed at a temperature between 20 and 30° C.

6. The method according to claim 5, wherein the substrate bed is provided with 25 to 400 sweet potato tubers per m².

7. The method according to claim 4, wherein the sweet potato tuber has been subjected, prior to growing, to a temperature between 27 and 40° C. and a relative humidity of at least 80% for a period of 4 to 16 days.

8. The method according to claim 1, wherein the method comprises rooting the cutting by providing the basal end of the stem section in a substrate material provided in a plug.

9. The method according to claim 8, wherein the plug is a substantially cylindrical plug with an open top and an open bottom.

10. The method according to claim 8, wherein the plug is substantially made of a biodegradable material.

11. The method according to claim 8, wherein the plug is substantially made of paper.

12. The method according to claim 1, wherein the method comprises removing unwanted leaves from the sweet potato plant, wherein an unwanted leaf has a length between a leaf base and an apical end of at least 3 cm.

13. The method according to claim 1, wherein the sweet potato plant cutting is taken by hand, scissors or a knife.

14. Sweet potato propagating material, wherein the propagating material is a cutting obtained from a growing sweet potato plant, wherein the cutting is a stem section provided with an apical bud, a basal end and at least one node and in which a length of the stem section between an apical end of the apical bud and the basal end is between 2 and 10 cm.

15. Sweet potato propagating material according to previous claim 14 obtained according to a method for obtaining a propagating material for sweet potato, the method comprising taking a cutting from a stem of a growing sweet potato plant, wherein the cutting is a stem section provided with an apical bud and at least one node and wherein a length of the stem section between an apical end of the apical bud and a basal end is between 2 and 10 cm.

16. A plurality of the sweet potato propagating material according to claim 14 packed in a bundle.

17. A plurality of the propagating material according to claim 16, wherein the packaging is a perforated film.
